(19) United States
(12) Patent Application Publication
(10) Pub. No.: US 2022/0306563 A1
(43) Pub. Date: Sep. 29, 2022

(54) METHOD FOR PREPARING GLYCOLIC ACID

(71) Applicant: RHODIA OPERATIONS, Aubervilliers (FR)

(72) Inventors: Zhen YAN, Shanghai (CN); Bright KUSEMA, Shanghai (CN); Stéphane STREIFF, Shanghai (CN)

(21) Appl. No.: 17/610,761

(22) PCT Filed: Jun. 27, 2019
(86) PCT No.: PCT/CN2019/093182

§ 371 Date: Nov. 12, 2021

(51) Int. Cl. C07C 51/235; B01J 23/644; B01J 23/42; B01J 21/18

(52) U.S. Cl. CPC C07C 51/235; B01J 23/6447; B01J 23/42; B01J 21/18

(57) ABSTRACT

Provided is a method for preparing glycolic acid which comprises oxidizing glycolaldehyde with molecular oxygen in the presence of a solvent and a supported catalyst. Said supported catalyst comprises (i) a noble metal selected from the group consisting of Pt, Pd, Ru and Rh, (ii) Bi and (iii) a support. Advantageously, the supported metallic catalyst is more active than the catalysts used in prior art. Furthermore, the catalyst is more stable at oxygen rich conditions.

### TECHNICAL FIELD

The present invention relates to a method for preparing glycolic acid comprising oxidizing glycolaldehyde with molecular oxygen in the presence of a solvent and a supported catalyst comprising (i) a noble metal selected from the group consisting of Pt, Pd, Ru and Rh, (ii) Bi and (iii) a support.

### BACKGROUND

Glycolic acid has conventionally been used mainly as boiler compounds, cleaning agents, leather tanning agents, chelating agents of metal ions and the like. In recent years, its applications have expanded to cosmetics, personal care and pharmaceuticals for external use. Glycolic acid to be used for pharmaceuticals requires high purity grade and is desired to contain a lower level of harmful impurities. Glycolic acid has recently been expected also as a raw material for polyglycolic acid having biodegradability and a gas barrier function.

Typical examples of a conventionally known method for producing glycolic acid include (1) a method of reacting carbon monoxide, formaldehyde and water in the presence of a strongly acidic catalyst under high-temperature and high-pressure conditions, (2) a method of reacting formaldehyde with hydrogen cyanide, (3) a method of reacting chloroacetic acid with sodium hydroxide, (4) a method of carrying out a Cannizzaro reaction between glyoxal available by oxidation of ethylene glycol and a strong alkali to form a glycolate salt, and then adding an acid to liberate glycolic acid from the resulting glycolate salt; (5) a method of carrying out a liquid-phase reaction between glyoxal available by oxidation of ethylene glycol and water in the presence of an inorganic catalyst; (6) a method for catalytic oxidation of ethylene glycol in the presence of a noble metal catalyst and oxygen; and (7) a method of carrying out oxidative esterification of ethylene glycol with methanol and oxygen to obtain methyl glycolate and then hydrolyzing into glycolic acid.

The method (1) is performed in the presence of a strongly acidic catalyst such as acidic polyoxometalate under high-temperature and high-pressure conditions. Thus, special reaction equipment and special reaction conditions of high temperature and high pressure are necessary. At the same time, glycolic acid obtained using reaction conditions of high temperature and high pressure contains a large amount of various impurities.

The method (2) of reacting formaldehyde with hydrogen cyanide requires the use of an extremely poisonous starting raw material, i.e., hydrogen cyanide.

The method (3) of reacting monochloroacetic acid with sodium hydroxide requires use of an about stoichiometric amount of sodium hydroxide. One problem is that sodium chloride generated raises the slurry concentration, leading to poor operability. Another problem is that this salt cannot be removed completely and remains in the product.

A problem common to the methods (4) to (7) is that ethylene glycol is produced from fossil-based feedstocks. For example, ethylene glycol can be produced using ethylene oxide as a raw material. The step of producing ethylene glycol is long and in addition, ethylene oxide, which is explosive, must be well handled in the production process.

As reported by Electrochimica Acta (1994), 39(11-12), 1877-80, previous efforts to oxidize glycolaldehyde have shown that the primary product from the electrochemical oxidation of glycolaldehyde over Pt electrodes is glyoxal, with only minor production of glycolic acid. Electrochemical modification of the electrode surface by deposition of an ad-atom layer of Bi was necessary to shift the selectivity to glycolic acid; a process which is not easily translated into industrial production.

The conventional production methods have the above-described drawbacks. In particular, glycolic acid obtained by these methods utilize fossil-based feedstocks.

U.S. Pub. No. 2013/0281733 reports glycolaldehyde was oxidized to glycolic acid using 0.5 MPa 02 at 180° C. in the presence of a molybdenum-containing acidic catalysts. Glycolaldehyde in this case was an intermediate in cellulose oxidation. The yield of glycolic acid obtained by this method is low.

PCT. Pub. No. WO2018/095973 teaches a method for preparing glycolic acid from glycolaldehyde in the presence of a metal-based catalyst. Said metal-based catalyst is selected from the group consisting of Pt, Pd and mixtures thereof. However, due to the poor activity of this catalyst, high catalyst to substrate loading is necessary according to Example 1.

There is still a need to develop an industrially applicable process to prepare glycolic acid with a high yield and selectivity based on inexpensive and sustainable feedstocks, such as bio-based materials with desired characteristics such as low cost, simple equipment, mild reaction conditions, ease of handle, which can overcome the drawbacks in prior arts. Specifically, the inventors have now found that the supported catalyst comprising (i) a noble metal selected from the group consisting of Pt, Pd, Ru and Rh, (ii) Bi and (iii) a support is more active than the metal catalysts used in prior art. Thus, the selectivity and the yield to glycolic acid can be well improved by using this kind of supported catalyst. Meanwhile, high catalyst to substrate loading is not necessary in the reaction. Furthermore, the catalyst is more stable at oxygen rich conditions.

### SUMMARY OF THE INVENTION

The present invention therefore pertains to a method for preparing glycolic acid comprising oxidizing glycolaldehyde with molecular oxygen in the presence of a solvent and a supported catalyst comprising (i) a noble metal selected from the group consisting of Pt, Pd, Ru and Rh, (ii) Bi and (iii) a support.

The invention also concerns a mixture comprising glycolaldehyde, molecular oxygen, a solvent and a supported catalyst comprising (i) a noble metal selected from the group consisting of Pt, Pd, Ru and Rh, (ii) Bi and (iii) a support.

### Definitions

Throughout the description, including the claims, the term “comprising one” should be understood as being synonymous with the term “comprising at least one”, unless otherwise specified, and “between” should be understood as being inclusive of the limits.

As used herein, the terminology “(Cn-Cm)” in reference to an organic group, wherein n and m are each integers, indicates that the group may contain from n carbon atoms to m carbon atoms per group.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

It is specified that, in the continuation of the description, unless otherwise indicated, the values at the limits are included in the ranges of values which are given.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### DETAILS OF THE INVENTION

Glycolaldehyde subject to molecular oxygen oxidation can be a bio-based raw material. Bio-based raw material refers to a product consisting of a substance, or substances, originally derived from living organisms. These substances may be natural or synthesized organic compounds that exist in nature. For example, it is known that glycolaldehyde can be produced by high-temperature fragmentation of carbohydrates to produce a mixture of Ci-C3 oxygenates such as described in U.S. Pat. Nos. 7,094,932, 5,397,582 and WO 2017/216311.

The carbohydrate used for thermal fragmentation to provide a Ci-C3 oxygenate mixture may be mono- and/or disaccharide. In an embodiment, the mono- and/or di-saccharide is selected from the group consisting of sucrose, lactose, xylose, arabinose, ribose, mannose, tagatose, galactose, glucose and fructose; or mixtures thereof. In a further embodiment, the monosaccharide is selected from the group consisting of glucose, galactose, tagatose, mannose, fructose, xylose, arabinose, ribose; or mixtures thereof.

As used herein, molecular oxygen is a diatomic molecule that is composed of two oxygen atoms held together by a covalent bond.

In one embodiment, molecular oxygen is supplied in the form of oxygen gas. Preferably, the purity of oxygen gas is of at least 99%. The oxidation reaction is performed at an O₂ partial pressure which is advantageously in the range of 1 to 10 bar in this embodiment.

In another embodiment, molecular oxygen is supplied in the form of air. The oxidation reaction is performed at an air partial pressure which is advantageously in the range of 0.15 to 1 bar in this embodiment.

The reaction may be carried out in a batch type reactor or in a continuous type reactor. In batch type reactor, the molar ratio of molecular oxygen to glycolaldehyde preferably ranges from 1 to 10 mol/mol. In continuous type reactor, the molecular oxygen flow rate preferably ranges from 0.1 to 0.5 L/min.

The noble metal in the supported catalyst is selected from the group consisting of Pt, Pd, Ru and Rh. Preferably, the noble metal is Pt.

The support to the metal catalyst is not particularly limited. It can notably be a metal oxide chosen in the group consisting of aluminum oxide (Al₂O₃), silicon dioxide (SiO₂), titanium oxide (TiO₂), zirconium dioxide (ZrO₂), calcium oxide (CaO), magnesium oxide (MgO), lanthanum oxide (La₂O₃), niobium dioxide (NbO₂), cerium oxide (CeO₂) and mixtures thereof.

The support can also be a zeolite. Zeolites are substances having a crystalline structure and a unique ability to change ions. People skilled in the art can easily understand how to obtain those zeolites by preparation method reported, such as zeolite L is described in U.S. Pat. No. 4,503,023 or commercial purchase, such as ZSM available from ZEOLYST.

The support of catalyst can even be Kieselguhr, clay or carbon.

Preferably, the support is carbon or aluminum oxide (Al₂O₃). More preferably, the support is carbon.

The loading of the noble metal ranges from 1 to 10 wt. % based on total weight of catalyst and preferably from 3 to 5 wt. %.

The weight ratio of Bi to the noble metal in the supported catalyst preferably ranges from 0.03 to 1 and more preferably from 0.2 to 0.3.

It was surprisingly found that the supported catalyst comprising (i) a noble metal selected from the group consisting of Pt, Pd, Ru and Rh, (ii) Bi and (iii) a support has better catalytic activity. Thus, the loading of catalyst to substrate can be lower than prior art to achieve the same performance. Preferable weight ratio of the supported catalyst to glycolaldehyde is from 5 to 50% and more preferably from 5 to 10%.

The supported catalysts used in the method according to the present invention include those commercially available, such as Pt-Bi/C from Johnson Matthey.

The solvent used in the method according to the present invention can be water, ether, methanol or ethanol. Preferable solvent is water.

The method according to the present invention comprises the following steps:

(i) Mixing glycolaldehyde, molecular oxygen, a solvent and a supported catalyst comprising (i) a noble metal selected from the group consisting of Pt, Pd, Ru and Rh, (ii) Bi and (iii) a support;

(ii) Heating the mixture obtained at step (i) at proper temperature for proper time to prepare glycolic acid.

The proper temperature can be preferably from 20 to 120° C.

The proper time can be preferably from 0.25 h to 25 h.

The invention also concerns a mixture comprising glycolaldehyde, molecular oxygen, a solvent and a supported catalyst comprising (i) a noble metal selected from the group consisting of Pt, Pd, Ru and Rh, (ii) Bi and (iii) a support.

The following examples are included to illustrate embodiments of the invention. Needless to say, the invention is not limited to described examples.

### EXPERIMENTAL PART

Materials
Glycolaldehyde Dimer, CAS No. 23147-58-2, purity >95% from Adamas-beta
5% Pt-1.5% Bi/C, Type 160, CAS No. 7440-06-4, Johnson Matthey
5% Pt/C, CAS No. 7440-06-4, Johnson Matthey

### Example 1

240 mg of glycolaldehyde, 2.0 mL of water and 25 mg of 5 wt. % Pt-1.5 wt. % Bi/C catalyst were added to a stainless-steel autoclave with a Teflon insert. The autoclave was closed and charged with 10 bar of oxygen. The autoclave was heated to 80° C., stirred using a magnetic stir bar and held for 6 hours. After reaction, the products were analyzed by HPLC. The conversion of glycolaldehyde was 97% and the yield to glycolic acid was 78%.

### Example 2

240 mg of glycolaldehyde, 1.5 mL of water and 50 mg of 5 wt. % Pt-1.5 wt. % Bi/C catalyst were added to a stainless-steel autoclave with a Teflon insert. The autoclave was closed and charged with 10 bar of oxygen. The autoclave was heated to 30° C., stirred using a magnetic stir bar and held for 24 hours. After reaction, the products were analyzed by HPLC. The conversion of glycolaldehyde was 83% and the yield to glycolic acid was 74%.

### Example 3

240 mg of glycolaldehyde, 1.5 mL of water and 50 mg of 5 wt. % Pt/C catalyst were added to a stainless-steel autoclave with a Teflon insert. The autoclave was closed and charged with 10 bar of oxygen. The autoclave was heated to 30° C., stirred using a magnetic stir bar and held for 24 hours. After reaction, the products were analyzed by HPLC. The conversion of glycolaldehyde was 72% and the yield to glycolic acid was 56%.

### Example 4

480 mg of glycolaldehyde, 4.0 mL of water and 50 mg of 5 wt. % Pt-1.5 wt. % Bi/C catalyst were added to a glass flask with a condenser. Air was bubbled through the liquid mixture at 0.1 L/min. The glass flask was heated to 60° C. and held for 7 hours. After reaction, the products were analyzed by HPLC. The conversion of glycolaldehyde was 82% and the yield to glycolic acid was 71%.

### Example 5

480 mg of glycolaldehyde, 4.0 mL of water and 150 mg of 5 wt. % Pt/C catalyst were added to a glass flask with a condenser. Air was bubbled through the liquid mixture at 0.1 L/min. The glass flask was heated to 60° C. and held for 7 hours. After reaction, the products were analyzed by HPLC. The conversion of glycolaldehyde was 18% and the yield to glycolic acid was 16%.

1. A method for preparing glycolic acid comprising oxidizing glycolaldehyde with molecular oxygen in the presence of a solvent and a supported catalyst comprising (i) a noble metal selected from the group consisting of Pt, Pd, Ru and Rh, (ii) Bi and (iii) a support.

2. The method according to claim 1, wherein the weight ratio of Bi to the noble metal in the supported catalyst ranges from 0.03 to 1.

3. The method according to claim 2, wherein the weight ratio of Bi to the noble metal in the supported catalyst ranges from 0.2 to 0.3.

4. The method according to claim 1, wherein the loading of the noble metal ranges from 1 to 10 wt. % based on total weight of catalyst.

5. The method according to claim 4, wherein the loading of the noble metal ranges from 3 to 5 wt. % based on total weight of catalyst.

6. The method according to claim 1, wherein the weight ratio of the supported catalyst to glycolaldehyde is from 5 to 50%.

7. The method according to claim 6, wherein the weight ratio of the supported catalyst to glycolaldehyde is from 5 to 10%.

8. The method according to claim 1, wherein the noble metal is Pt.

9. The method according to claim 1, wherein the support is carbon or aluminum oxide.

10. The method according to claim 1, wherein molecular oxygen is supplied in the form of oxygen gas or air.

11. The method according to claim 10, wherein molecular oxygen is supplied in the form of oxygen gas having a purity of at least 99%.

12. A mixture comprising glycolaldehyde, molecular oxygen, a solvent and a supported catalyst comprising (i) a noble metal selected from the group consisting of Pt, Pd, Ru and Rh, (ii) Bi and (iii) a support.

13. The mixture according to claim 12, wherein molecular oxygen is in the form of oxygen gas having a purity of at least 99%.

14. The mixture according to claim 12, wherein the solvent is water.

15. The mixture according to claim 12, wherein the noble metal is Pt.

16. The mixture according to claim 12, wherein the support is carbon.
